(19) United States
(12) Patent Application Publication
(10) Pub. No.: US 2023/0268080 A1
(43) Pub. Date: Aug. 24, 2023

(54) ANALYSIS SYSTEM AND METHOD FOR CAUSAL INFERENCE OF DIGITAL THERAPEUTICS BASED ON MOBILE DATA

(71) Applicant: Korea Advanced Institute of Science and Technology, Daejeon (KR)

(72) Inventors: Uichin LEE, Daejeon (KR); Gyuwon JUNG, Daejeon (KR); Eun-Yeol MA, Daejeon (KR); Heeyoung KIM, Daejeon (KR)

(21) Appl. No.: 17/844,648

(22) Filed: Jun. 20, 2022

(30) Foreign Application Priority Data
Feb. 24, 2022 (KR) 10-2022-0024541

(51) Int. Cl. G16H 50/70

(52) U.S. Cl. CPC G16H 50/70

(57) ABSTRACT

Provided is an analysis system and method for causal inference of digital therapeutics based on mobile data. An analysis method for causal inference of digital therapeutics performed by an analysis system may include collecting mobile data generated by a mobile device; performing a causal inference of digital therapeutics using the collected mobile data; and providing an analysis result acquired through the performed causal inference of the digital therapeutics.

### CROSS-REFERENCE TO RELATED APPLICATION

This application claims the priority benefit of Korean Patent Application No. 10-2022-0024541, filed on Feb. 24, 2022, in the Korean Intellectual Property Office, the disclosure of which is incorporated herein by reference.

### BACKGROUND

### 1. Field of the Invention

The following description of example embodiments relates to technology for inferring and analyzing causality of digital therapeutics based on mobile data.

### 2. Description of the Related Art

Digital therapeutics (DTx) refers to a high-quality software program that provides evidence-based therapeutic intervention and is defined as having the purpose of preventing, managing, and treating disorders or diseases. The digital therapeutics is used alone or with other therapeutic techniques and equipment to care for patients or to optimize their health status. In Korea, the Ministry of Food and Drug Safety also defines a software medical device applied for prevention, management, and treatment purposes of diseases and also having a clinical basis as a “digital treatment device”. Related to this, the United States Food and Drug Administration also provides guidelines for Software as a Medical Device for accelerating the high-quality digital health innovation, together with validation processes such as the Pre-Cert Program to ensure the evidence (i.e., effectiveness in relieving target symptoms) of health-related software before using it for medical purposes.

To be used as digital therapeutics, therapeutic effect based on scientific evidence and clinical trials to prove the same are essential and, similar to over-the-counter drugs and therapeutics, approval from the licensing authority is required based on medical evidence. Therefore, to prove the therapeutic effect of digital therapeutics, a difference in significant symptoms between a group (an experimental group) that used the digital therapeutics and a group (control group) that did not use the same is generally presented as evidence using a randomized controlled trial (RCT). In the RCT, since experimental subjects are randomly assigned to two groups, it is possible to assume that all external variables related to use of digital therapeutics are well controlled and, finally, the causality of digital therapeutics is proved by comparing levels of symptoms (outcomes) that digital therapeutics desire to address.

However, unlike the laboratory settings, there are several practical limitations when verifying the causality of digital therapeutics with random assignment of subjects in the real world. The random assignment of experimental subjects is limited in terms of cost and time since it requires the recruitment of a large number of subjects for the validity. In the case of real-world data (RWD), which is collected outside the laboratory, there are several external variables that may affect the use and effect of digital therapeutics since they cannot be perfectly controlled. If all of these variables are not controlled in a randomization scheme, they will become confounding factors that lead to a wrong conclusion about the causal relationship. In addition, various detailed elements and functions can be implemented even within one digital therapeutics, and their usage patterns can vary depending on the patient. Therefore, it becomes more difficult to analyze exactly which element or function of the digital therapeutics is responsible for relieving target symptoms of a patient.

### SUMMARY

Example embodiments may provide a system and method for automatically analyzing and verifying causality of digital therapeutics using mobile data even in an environment in which a randomized controlled trial (RCT) is limited.

According to an aspect of example embodiments, there is provided an analysis method for causal inference of digital therapeutics performed by an analysis system, the analysis method including collecting mobile data generated by a mobile device; performing a causal inference of digital therapeutics using the collected mobile data; and providing an analysis result acquired through the performed causal inference of the digital therapeutics.

The collecting may include collecting digital therapeutics data that includes interaction data of the digital therapeutics, target behavior data of performing a target behavior suggested by the digital therapeutics, and target symptom data for a target symptom to be controlled by the digital therapeutics.

The collecting may include collecting internal data that includes interaction data of a mobile device including screen ON/OFF of the mobile device, an application use, a call use, and a short messaging service (SMS) use and sensor data in the mobile device.

The collecting may include collecting external data that includes external service data provided through an external application programming interface (API) and external measurement data of an external measurement device.

The collecting may include collecting user data that includes user identification data or user subjective evaluation data.

The performing may include extracting variable data representing a status and a behavior of a user using the collected mobile data.

The performing may include generating a data point of a basic unit for the extracted variable data and generating a processed dataset by synthesizing the generated data point.

The performing may include converting to the variable data for verifying the status and the behavior of the user using the collected mobile data and extracting, from the converted variable data, variable data within a time unit of a desired interval or a pre-specified time interval.

The performing may include excluding a variable unnecessary for causal inference in response to a section of a cause variable and an outcome variable from the extracted variable data and controlling an external variable that affects the causal inference.

The performing may include performing the causal inference according to the selected cause variable and outcome variable and verifying a validity for a result of the performed causal inference.

The performing may include verifying whether a change in the cause variable affects a change in the outcome variable through a correlation analysis between cause data and outcome data while controlling and correcting the external variable that affects the causal inference through a potential outcome framework.

The providing may include allowing a user to confirm the collected mobile data in a process of performing the causal inference on the collected mobile data and establishing a hypothesis for the causal inference the mobile data confirmed by the user.

The providing may include providing an analysis result acquired through the performed causal inference of the digital therapeutics in a form of a text, a table, or a graph.

The providing may include proposing a new causal inference to perform causal inference between other variables related to a cause variable or an outcome variable selected for the causal inference.

According to an aspect of example embodiments, there is provided a non-transitory computer-readable recording medium storing instructions that, when executed by a processor, cause the processor to perform an analysis method for causal inference of digital therapeutics performed by an analysis system, the analysis method including collecting mobile data generated by a mobile device; performing a causal inference of digital therapeutics using the collected mobile data; and providing an analysis result acquired through the performed causal inference of the digital therapeutics.

According to an aspect of example embodiments, there is provided an analysis system for causal inference of digital therapeutics, the analysis system including a mobile data collector configured to collect mobile data generated by a mobile device; a mobile data analyzer configured to perform a causal inference of digital therapeutics using the collected mobile data; and a mobile data visualizer configured to providing an analysis result acquired through the performed causal inference of the digital therapeutics.

According to some example embodiments, it is possible to automatically analyze and verify causality of digital therapeutics for a specific behavior or symptom by collecting mobile data, by extracting a feature variable capable of expressing daily life of a patient, and by building a causal inference environment similar to an RCT through a selection of the extracted feature variable. Therefore, it is possible to verify effect of digital therapeutics even in an environment in which the RCT is limited.

According to some example embodiments, when applying and analyzing daily life data of a user, it is possible to automatically infer a cause that triggers a specific behavior of the user, a cause that places the user to be in a specific status, and the like.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates an example of an analysis operation for causal inference of digital therapeutics according to an example embodiment;

FIG. 2 is a diagram illustrating an example of a configuration of an analysis system according to an example embodiment;

FIG. 3 is a flowchart illustrating an example of an analysis method for causal inference of digital therapeutics by an analysis system according to an example embodiment;

FIG. 4 illustrates an example of a mobile data collecting operation according to an example embodiment;

FIG. 5 illustrates an example of a mobile data processing operation according to an example embodiment;

FIG. 6 illustrates an example of a variable selecting operation according to an example embodiment; and

FIG. 7 illustrates an example of a causal inference operation according to an example embodiment.

### DETAILED DESCRIPTION

Hereinafter, some example embodiments will be described in detail with reference to the accompanying drawings. The following detailed structural or functional description of example embodiments is provided as an example only and various alterations and modifications may be made to the example embodiments. Accordingly, the example embodiments are not construed as being limited to the disclosure and should be understood to include all changes, equivalents, and replacements within the technical scope of the disclosure.

The terminology used herein is for describing various example embodiments only, and is not to be used to limit the disclosure. The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Terms, such as first, second, and the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component, without departing from the scope of the disclosure.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Regarding the reference numerals assigned to the elements in the drawings, it should be noted that the same elements will be designated by the same reference numerals, wherever possible, even though they are shown in different drawings. Also, in the description of embodiments, detailed description of well-known related structures or functions will be omitted when it is deemed that such description will cause ambiguous interpretation of the present disclosure.

Hereinafter, example embodiments are described with reference to the accompanying drawings.

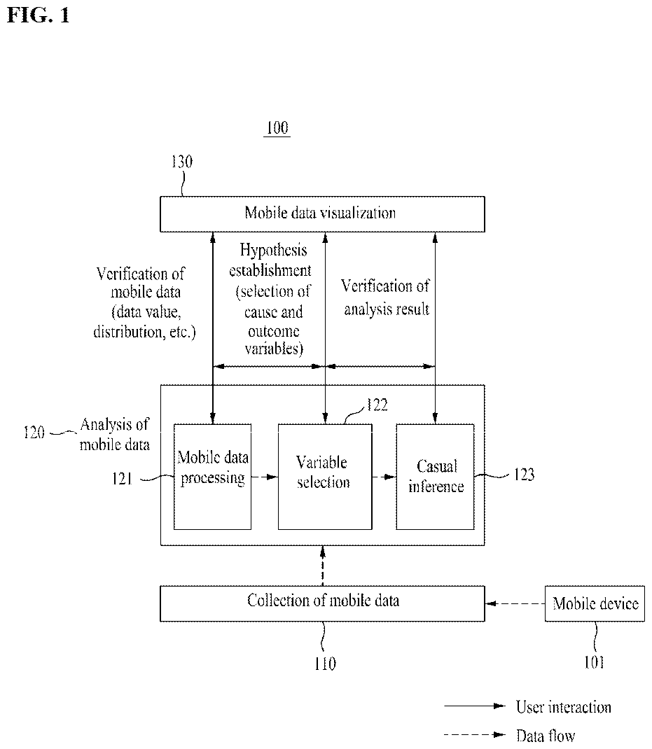

FIG. 1 illustrates an example of an analysis operation for causal inference of digital therapeutics according to an example embodiment.

An analysis system 100 may automatically analyze and verify causality of digital therapeutics using mobile data generated by a mobile device 101.

The analysis system 100 may collect the mobile data generated by the mobile device 101 (110). Here, the mobile device 101 may include, for example, a portable device, such as a smartphone, a mobile phone, a navigation device, a computer, a laptop computer, a digital broadcasting terminal, a personal digital assistant (PDA), a portable multimedia player (PMP), a tablet personal computer (PC), a game console, a wearable device, an Internet of things (IoT) device, a virtual reality (VR) device, an augmented reality (AR) device, and the like. For example, the analysis system 100 may collect mobile data of a patient using the mobile device 101.

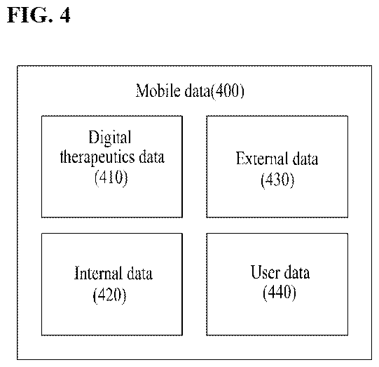

FIG. 4 illustrates an example of a mobile data collecting operation according to an example embodiment. Referring to FIG. 4, the analysis system 100 may collect mobile data 400 that includes digital therapeutics data 410, internal data 420, external data 430, and user data 440. In an example embodiment, a causal inference of digital therapeutics that assists weight loss of a patient through promotion of a physical activity is described using an example. To this end, description is made with assumption of a causal inference scenario for “Has an activity of viewing a diet management video in digital therapeutics caused weight loss?”.

In more detail, the analysis system 100 may collect the digital therapeutics data 410 that includes interaction data of the digital therapeutics, target behavior data of performing a target behavior suggested by the digital therapeutics, and target symptom data for a target symptom to be controlled by the digital therapeutics. The analysis system 100 may collect digital interaction data related to a digital therapeutics usage record of a patient, a preemptive interaction (e.g., a notification message) of the digital therapeutics, and the like. Here, various detailed elements may be present in the digital therapeutics and the analysis system 100 may collect a usage record and pattern for each detailed element. For example, the analysis system 100 may collect a usage record and pattern for each detailed element, such as a health status/digital therapeutics usage record (e.g., health status information of a patient such as a weight, a blood pressure, etc., and a point in time at which the digital therapeutics is executed), health-related learning materials (e.g., a diet management video, a posting on a stretching method, etc.), a digital therapeutics usage comparison with other patients (e.g., a health-related video viewing rate of other patients, a goal achievement rate of other patients for a number of steps, and a number of executions of the digital therapeutics, etc.). Also, the analysis system 100 may collect data (e.g., a number of stretching during work) related to performing a specific target behavior suggested by the digital therapeutics. Also, the analysis system 100 may collect data (e.g., a weight) related to a specific target symptom that the digital therapeutics desires to finally control.

The analysis system 100 may include the internal data 420 generated in a mobile device, such as interaction data of the mobile device including screen ON/OFF of the mobile device, an application use, a call use, and a short messaging service (SMS) use and sensor data (e.g., an accelerometer, a global positioning system (GPS), Bluetooth, wireless fidelity (Wi-Fi), a microphone) in the mobile device.

The analysis system 100 may collect the external data 430 delivered to the mobile device, such as external service data provided through an external application programming interface (API) and external measurement data of an external measurement device. The analysis system 100 may collect the external service data (e.g., weather) delivered to the mobile device from a web service through the external API. The analysis system 100 may collect the external measurement data (e.g., a height, a weight, and a blood pressure) delivered from the external measurement device (e.g., InBody, a scale, a blood pressure measuring device) to the mobile device.

The analysis system 100 may collect the user data 440 that includes user identification data or user subjective evaluation data. The analysis system 100 may include the user identification data (basic user information) such as gender, age, occupation, personality, and disease/disorder status. The analysis system 100 may collect subjective evaluation data such as emotion and a stress state of the user. Here, the user data 440 may be directly input from the user to the mobile device.

The analysis system 100 may collect the mobile data 400 that includes the digital therapeutics data 410. The analysis system 100 may store a generation point in time (timestamp) of the mobile data 400, a type and a value of the mobile device and data, and the like.

The analysis system 100 may perform a mobile data analysis 120 of the collected mobile data 400. The analysis system 100 may perform a causal inference based on the collected mobile data 400.

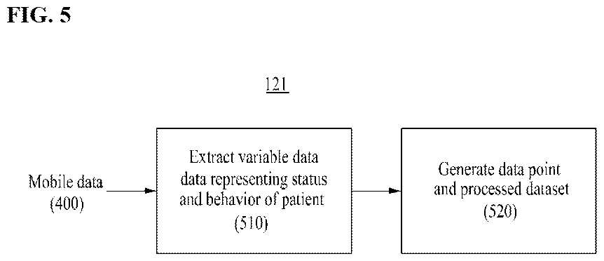

FIG. 5 illustrates an example of a mobile data processing operation according to an example embodiment. Here, FIG. 5 illustrates a mobile data processing 121 of FIG. 1. In operation 510, the analysis system 100 may extract variable data (value) representing a status and a behavior of a patient by processing collected mobile data, for example, the mobile data 400. The analysis system 100 may perform a data preprocessing process. The analysis system 100 may process a missing value and an outlier for the collected mobile data and may exclude data of the patient that affects data reliability. Here, processing of the missing value represents removing the missing value for the collected mobile data or replacing the missing value with an average, a median, or a mode (a categorical type). Also, processing of the outlier represents removing the outlier of the collected mobile data using a method, such as a standard score, an interquartile range (IQR), and the like.

The analysis system 100 may convert collected raw mobile data to a variable capable of verifying a behavior and context of the patient. For example, the collected raw mobile data may be converted to a physical activity (walking, stationary, etc.), a location (home, work, etc.), a sleep state (physical activity+location+time of day), presence or absence with a nearby person (location+ambient sound+Bluetooth), and work concentration (smartphone usage+location+time of day), in relation to the patient.

Since the collected mobile data is based on time series data, the analysis system 100 may extract a variable value based on a desired interval of a time unit to perform a causal inference. The analysis system 100 may set a different time interval according to a detailed element of digital therapeutics to be analyzed or a behavior goal of the digital therapeutics. For example, in the case of digital therapeutics that requires a user to perform an immediate target behavior, the analysis system 100 may set a short section in units of a minute. If it takes a long time for the user to acquire and recognize new health-related information from the digital therapeutics and then actually perform a target behavior, the analysis system 100 may set a long section in units of hour or day. When the existing collected mobile data is present, the analysis system 100 may search for an optimal section size by learning an average time interval used from use of the digital therapeutics to performing of a target behavior and may generate a data point according to the found optimal section size. The analysis system 100 may extract a variable value within a designated time section based on the collected mobile data. For example, the variable value may include a sum, an average, a standard deviation, a number, a maximum value, a minimum value, etc., within a time section according to a type. Here, the variable value may vary over time and may be constant at all times, such as a basic characteristic (e.g., gender, age, etc.) of the user.

In operation 520, the analysis system 100 may generate a data point of a basic unit and a processed dataset. The analysis system 100 may define a collection of a plurality of variable values within a single time section as a single data point. Here, the data point may be data of a basic unit used to analyze the causal inference. The data point may be generated based on a point in time at which a specific event occurs according to a causal relationship to be analyzed (e.g., a point in time at which a notification message is provided to the user in the digital therapeutics) and may be generated based on a desired time interval regardless of occurrence of an event (e.g., on an hourly basis). The analysis system 100 may generate a single processed dataset by synthesizing data points.

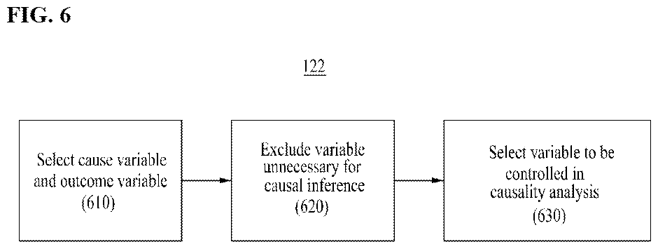

Referring again to FIG. 1, the analysis system 100 may perform a variable selection 122 of selecting a variable from the collected mobile data. FIG. 6 illustrates an example of a variable selecting operation according to an example embodiment. Here, FIG. 6 illustrates the variable selection 122 of FIG. 1. According to a selection of a cause variable and an outcome variable, the analysis system 100 may exclude a variable unnecessary for a causal inference and may select a variable to be controlled in a causal inference process.

Referring to FIG. 6, in operation 610, the analysis system 100 may select a cause variable and an outcome variable. For example, the analysis system 100 may provide a user interface for a variable selection from a user. In response thereto, the user may select the cause variable and the outcome variable through the provided user interface. The analysis system 100 may receive the selected cause variable and outcome variable from the user. Alternatively, the analysis system 100 may automatically select the cause variable and the outcome variable. The cause variable refers to using a variable corresponding to interaction data of the digital therapeutics and the outcome variable refers to using a variable corresponding to target behavior data or target symptom data of the digital therapeutics. For example, in an example embodiment, “viewing time of a diet management video in digital therapeutics” may be used as the cause variable and “weight” may be used as the outcome variable. Here, the outcome variable needs to use a value that occurs later than the cause variable in time order. That is, if a value of the cause variable occurs at a time t, the outcome needs to occur at a time t+a (a>0).

In operation 620, in response to the selection of the cause variable and the outcome variable, the analysis system 100 may exclude a variable unnecessary for the causal inference. When variables have the same value, the analysis system 100 may exclude a variable unnecessary for the causal inference. For example, a variable having a variance less than a desired value based on a variance value of the variable may be excluded from analysis (e.g., in a case in which almost all values are zeroes).

In operation 630, the analysis system 100 may select a variable to be controlled in the causal inference process. The analysis system 100 may control an external variable that may cause a bias in the causal inference process. The analysis system 100 may control the external variable that may distort a causal result by affecting a cause and an outcome to perform the causal inference. A representative example of the external variable may include a confounding variable. A related variable may be extracted at the same time with the cause variable and the outcome variable. The confounding variable refers to a variable having a significant correlation (i.e., related at the same time) to each of the cause variable and the outcome variable by at least a predetermined criterion. The confounding variable needs to take precedence over the outcome variable in time order and a variable group to be controlled may be selected by adjusting a correlation criterion to be controlled according to a degree (intensity) of confounding.

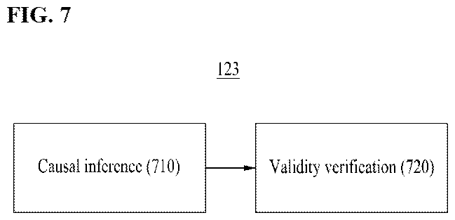

Referring again to FIG. 1, the analysis system 100 may perform a causal inference of digital therapeutics for a target behavior and symptom based on a variable and may verify validity for a result of the performed causal inference. FIG. 7 illustrates a causal inference operation according to an example embodiment. Here, FIG. 7 illustrates the causal inference 123 of FIG. 1. Referring to FIG. 7, in operation 710, the analysis system 100 may perform a causal inference. A causal inference method may use various methods. As a representative method, a potential outcome framework may be used. Through the potential outcome framework, the analysis system 100 may select an external variable that causes a bias and then analyze a correlation between cause data and outcome data while controlling and correcting the selected external variable and may verify whether a change in the cause variable has a significant effect on a change in the outcome variable. The purpose of use of digital therapeutics may be classified into a treated group when used by more than a specific criterion and may be classified into a control group when less used. Each data point may be assigned to one of two groups according to a criterion set for classifying the purpose of use of digital therapeutics. For example, based on an average value of “viewing time of a diet management video in digital therapeutics” in mobile data collected from specific individual patients, the analysis system 100 may classy the patients into the treated group when viewing above the average and into the control group when viewing less.

When the external variable that causes the bias is present, the analysis system 100 may control the external variable using an algorithm of sampling data points such that the external variable is distributed similarly between two groups and may set a difference only in the purpose of use (cause variable) of the digital therapeutics. The analysis system 100 may control the external variable using a machine learning method, such as genetic matching or using a distance between data points such as propensity score or Mahalanobis distance in a sampling process.

Accordingly, the analysis system 100 may perform a statistical analysis regarding whether a significant difference is present in an outcome variable between two groups and, when the significant difference is present, may infer that there is the causality of digital therapeutics for the outcome variable. That is, the difference in the result according to the causal inference is caused by the use of digital therapeutics. In an example embodiment, the causal inference may be performed based on whether the significant difference is present in a value of “weight.” In a stage of correcting the external variable, the difference may occur in a result of classifying a data point according to an algorithm and may affect a result. Therefore, it is possible to cross-validate a result of the causal inference through various algorithms and to improve reliability of the result. Also, although the analysis system 100 may perform the causal inference based on data of a single patient, the analysis system 100 may perform an inference by synthesizing data of a plurality of patients showing similar statuses and behaviors.

In operation 720, the analysis system 100 may perform a validity verification on the result of the causal inference. The analysis system 100 may validate the effect on causality of the cause variable and may use Neyman's null, Fisher's null, etc., based on an effect size. The analysis system 100 may verify the validity of the result by determining whether the same result is acquired with a dataset in which a portion of a dataset used in the causal inference is removed, or by determining whether a different result is acquired from that when the cause variable is substituted with another variable.

Referring to 130 of FIG. 1, the analysis system 100 may visualize an analysis result acquired through the causal inference of digital therapeutics. The analysis system 100 allows the user to verify mobile data in a process of analyzing the collected mobile data and may establish a hypothesis for the causal inference, may support a process of confirming an analysis result, and may propose a new causal inference.

The analysis system 100 may verify a type and a value of collected data and may also verify mobile data in various forms, such as a table, a graph, and the like. The analysis system 100 may establish a hypothesis for the causal inference. The user may select the cause variable and the outcome variable for the causal inference through the analysis system 100. The analysis system 100 may recommend the cause variable and the outcome variable and may perform the causal inference by selecting, by the user, the recommended cause variable and outcome variable. The analysis system 100 may exclude an unnecessary variable and may select a variable to be controlled according to the selected cause and outcome variables.

The analysis system 100 may verify a result of the performed causal inference. The analysis system 100 may perform the causal inference on the cause variable and the outcome variable selected from the user and may provide a final causality result when the validity of the result is proved. For example, the analysis system 100 may provide a causality result in various forms, such as a table and a graph. The analysis system 100 may provide a description related to a process of inferring the causality result using a text, a graph, and the like.

The analysis system 100 may propose a new causal inference. The analysis system 100 may recommend a causal inference between other variables related to a cause variable or an outcome variable directly selected by the user. That is, the analysis system 100 may guide the user to perform the causal inference between the other variables.

The analysis system 100 may be provided to verify another mobile data even after verifying the analysis result of the causal inference or to select another cause/outcome variable.

According to an example embodiment, since it is possible to extract a feature variable capable of expressing daily life of a patient by collecting mobile data, to build a causal inference environment similar to a randomized control trial (RCT) through a variable selection, and to automatically analyze and verify causality of digital therapeutics on a specific behavior or symptom based on the built causal inference environment, it is possible to verify effect of digital therapeutics even in an environment in which the RCT is limited.

Also, when applying and analyzing daily life data (e.g., a location, a physical activity, a smartphone use, an emotion, etc.) of a user, it is possible to automatically infer a cause that triggers a specific behavior of the user and a cause that places the user in a specific state. The analysis system 100 performs the causal inference based on observational data and thus, has a great use potent in that the analysis system 100 may perform the causal inference without a separate setting before experiment for the causal inference, such as a random assignment of an experiment subject. In addition, since the analysis system 100 uses mobile data, the analysis system 100 may be used in a process of exploring a cause of a specific behavior or state the user is unaware of before.

Through this, it is possible to verify the effect of digital therapeutics in a daily environment of a patient and to effectively verify validity by performing a causal inference based on data of the patient.

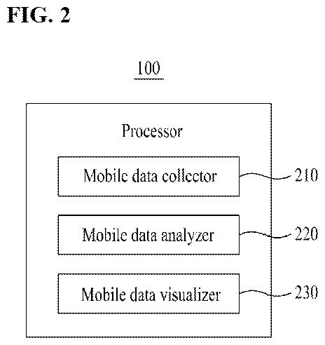

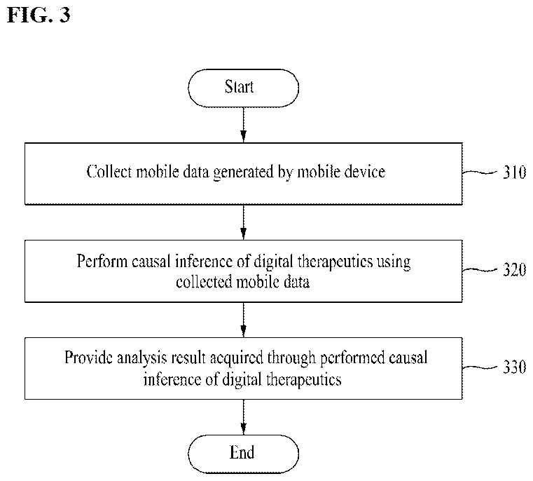

FIG. 2 is a diagram illustrating an example of a configuration of an analysis system according to an example embodiment, and FIG. 3 is a flowchart illustrating an example of an analysis method for causal inference of digital therapeutics by an analysis system according to an example embodiment.

A processor of the analysis system 100 may include a mobile data collector 210, a mobile data analyzer 220, and a mobile data visualizer 230. Components of the processor may be representations of different functions performed by the processor in response to a control instruction provided from a program code stored in the analysis system 100. The processor and the components of the processor may control the analysis system 100 to perform operations 310 to 330 included in the analysis method for causal inference of digital therapeutics of FIG. 3. Here, the processor and the components of the processor may be implemented to execute an instruction according to a code of at least one program and a code of an OS included in a memory.

The processor may load, to the memory, a program code stored in a file of a program for the analysis method for the causal inference of digital therapeutics. For example, in response to execution of the program on the analysis system 100, the processor may control the analysis system 100 to load the program code from the file of the program to the memory under control of the OS. Here, the mobile data collector 210, the mobile data analyzer 220, and the mobile data visualizer 230 may be different functional representations of the processor to perform the following operations 310 to 330 by executing an instruction of a portion corresponding to the program code loaded to the memory.

Referring to FIG. 3, in operation 310, the mobile data collector 210 may collect mobile data generated by a mobile device. The mobile data collector 210 may collect digital therapeutics data that includes interaction data of the digital therapeutics, target behavior data of performing a target behavior suggested by the digital therapeutics, and target symptom data for a target symptom to be controlled by the digital therapeutics. The mobile data collector 210 may collect internal data that includes interaction data of the mobile device including screen ON/OFF of the mobile device, an application use, a call use, and a short messaging service (SMS) use and sensor data in the mobile device. The mobile data collector 210 may collect external data that includes external service data provided through an external application programming interface (API) and external measurement data of an external measurement device. The mobile data collector 210 may collect user data that includes user identification data or user subjective evaluation data.

In operation 320, the mobile data analyzer 220 may perform a causal inference of digital therapeutics using the collected mobile data. The mobile data analyzer 220 may extract variable data representing a status and a behavior of a user using the collected mobile data. The mobile data analyzer 220 may generate a data point of a basic unit for the extracted variable data and generate a processed dataset by synthesizing the generated data point. The mobile data analyzer 220 may convert to variable data for verifying the status and the behavior of the user using the collected mobile data and may extract, from the converted variable data, variable data within a time unit of a desired interval or a pre-specified time interval. The mobile data analyzer 220 may exclude a variable unnecessary for causal inference in response to a section of a cause variable and an outcome variable from the extracted variable data and may control an external variable that affects the causal inference. The mobile data analyzer 220 may perform the causal inference according to the selected cause variable and outcome variable and may verify a validity for a result of the performed causal inference. The mobile data analyzer 220 may verify whether a change in the cause variable affects a change in the outcome variable through a correlation analysis between cause data and outcome data while controlling and correcting the external variable that affects the causal inference through a potential outcome framework.

In operation 330, the mobile data visualizer 230 may provide an analysis result acquired through the performed causal inference of the digital therapeutics. The mobile data visualizer 230 may allow the user to confirm the collected mobile data in a process of performing the causal inference on the collected mobile data and may establish a hypothesis for the causal inference the mobile data confirmed by the user. The mobile data visualizer 230 may provide an analysis result acquired through the performed causal inference of the digital therapeutics in a form of a text, a table, or a graph. The mobile data visualizer 230 may propose a new causal inference to perform causal inference between other variables related to the cause variable or the outcome variable selected for the causal inference.

The systems and/or apparatuses described herein may be implemented using hardware components, software components, and/or a combination thereof. For example, apparatuses and components described herein may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. A processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that the processing device may include multiple processing elements and/or multiple types of processing elements. For example, the processing device may include multiple processors or a processor and a controller. In addition, different processing configurations are possible, such as parallel processors.

The software may include a computer program, a piece of code, an instruction, or some combinations thereof, for independently or collectively instructing or configuring the processing device to operate as desired. Software and/or data may be embodied permanently or temporarily in any type of machine, component, physical equipment, virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable storage mediums.

The methods according to the example embodiments may be recorded in non-transitory computer-readable media including program instructions to implement various operations embodied by a computer. Also, the media may include, alone or in combination with the program instructions, data files, data structures, and the like. Program instructions stored in the media may be those specially designed and constructed for the purposes, or they may be of the kind well-known and available to those having skill in the computer software arts. Examples of non-transitory computer-readable media include magnetic media such as hard disks, floppy disks, and magnetic tape; optical media such as CD ROM disks and DVD; magneto-optical media such as floptical disks; and hardware devices that are specially to store and perform program instructions, such as read-only memory (ROM), random access memory (RAM), flash memory, and the like. Examples of program instructions include both machine code, such as produced by a compiler, and files containing higher level code that may be executed by the computer using an interpreter.

While this disclosure includes specific example embodiments, it will be apparent to one of ordinary skill in the art that various alterations and modifications in form and details may be made in these example embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed:

1. An analysis method for causal inference of digital therapeutics performed by an analysis system, the analysis method comprising:
collecting mobile data generated by a mobile device;
performing a causal inference of digital therapeutics using the collected mobile data; and
providing an analysis result acquired through the performed causal inference of the digital therapeutics.

2. The analysis method of claim 1, wherein the collecting comprises:
collecting digital therapeutics data that includes interaction data of the digital therapeutics, target behavior data of performing a target behavior suggested by the digital therapeutics, and target symptom data for a target symptom to be controlled by the digital therapeutics.

3. The analysis method of claim 1, wherein the collecting comprises collecting internal data that includes interaction data of a mobile device including screen ON/OFF of the mobile device, an application use, a call use, and a short messaging service (SMS) use and sensor data in the mobile device.

4. The analysis method of claim 1, wherein the collecting comprises collecting external data that includes external service data provided through an external application programming interface (API) and external measurement data of an external measurement device.

5. The analysis method of claim 1, wherein the collecting comprises collecting user data that includes user identification data or user subjective evaluation data.

6. The analysis method of claim 1, wherein the performing comprises extracting variable data representing a status and a behavior of a user using the collected mobile data.

7. The analysis method of claim 6, wherein the performing comprises generating a data point of a basic unit for the extracted variable data and generating a processed dataset by synthesizing the generated data point.

8. The analysis method of claim 6, wherein the performing comprises converting to the variable data for verifying the status and the behavior of the user using the collected mobile data and extracting, from the converted variable data, variable data within a time unit of a desired interval or a pre-specified time interval.

9. The analysis method of claim 6, wherein the performing comprises excluding a variable unnecessary for causal inference in response to a section of a cause variable and an outcome variable from the extracted variable data and controlling an external variable that affects the causal inference.

10. The analysis method of claim 9, wherein the performing comprises:
performing the causal inference according to the selected cause variable and outcome variable and verifying a validity for a result of the performed causal inference.

11. The analysis method of claim 10, wherein the performing comprises verifying whether a change in the cause variable affects a change in the outcome variable through a correlation analysis between cause data and outcome data while controlling and correcting the external variable that affects the causal inference through a potential outcome framework.

12. The analysis method of claim 1, wherein the providing comprises allowing a user to confirm the collected mobile data in a process of performing the causal inference on the collected mobile data and establishing a hypothesis for the causal inference the mobile data confirmed by the user.

13. The analysis method of claim 12, wherein the providing comprises providing an analysis result acquired through the performed causal inference of the digital therapeutics in a form of a text, a table, or a graph.

14. The analysis method of claim 13, wherein the providing comprises proposing a new causal inference to perform causal inference between other variables related to a cause variable or an outcome variable selected for the causal inference.

15. A non-transitory computer-readable recording medium storing instructions that, when executed by a processor, cause the processor to perform an analysis method for causal inference of digital therapeutics performed by an analysis system, the analysis method comprising:
collecting mobile data generated by a mobile device;
performing a causal inference of digital therapeutics using the collected mobile data; and
providing an analysis result acquired through the performed causal inference of the digital therapeutics.

16. An analysis system for causal inference of digital therapeutics, the analysis system comprising:
a mobile data collector configured to collect mobile data generated by a mobile device;
a mobile data analyzer configured to perform a causal inference of digital therapeutics using the collected mobile data; and
a mobile data visualizer configured to providing an analysis result acquired through the performed causal inference of the digital therapeutics.
